(19) United States
(12) Patent Application Publication
(10) Pub. No.: US 2010/0286530 A1
(43) Pub. Date: Nov. 11, 2010

(54) Photodynamic-based tissue sensing device and method

(76) Inventors: Saurav Paul, Minneapolis, MN (US); Israel Byrd, Richfield, MN (US)

Correspondence Address: SJM/AFD - DYKEMA;c/o CPA Global, P.O. Box 52050, Minneapolis, MN, 55402, US

(21) Appl. No.: 12/744,201

(22) PCT Filed: Dec. 18, 2008
(86) PCT No.: PCT/US08/87426

§ 371 Date: May 21, 2010

Related U.S. Application Data
(60) Provisional application No. 61/014,866, filed on Dec. 19, 2007.

(51) Int. Cl. A61B 6/00

(52) U.S. Cl. 600/478

(57) ABSTRACT

A system and method for diagnosis or treatment of tissue is provided. One or more optic fibers are disposed within a deformable, tubular body. An electronic control unit activates an electromagnetic radiation source to direct a first set of electromagnetic radiation through a first optic fiber to the tissue. The unit also receives a signal generated by an electromagnetic radiation sensor in response to a second set of electromagnetic radiation received through the first optic or a second fiber. The second set of electromagnetic radiation originates from the tissue in response to the first set of electromagnetic radiation and may be reflected or emitted by a substance contained in the tissue that alters radiation characteristics of the tissue. Finally, the unit is configured to determine a characteristic of the tissue (e.g. the distance from the tissue to the tubular body) responsive to the signal.

### BACKGROUND OF THE INVENTION

a. Field of the Invention

This invention relates to a system and method for diagnosis and/or treatment of tissue. In particular, the instant invention relates to a system and method in which electromagnetic radiation reflected or emitted from tissue is used to determine characteristics of the tissue during diagnosis and/or treatment.

b. Background Art

It is well known to use catheters in the diagnosis and treatment of tissues within a body. Catheters may be inserted within a vessel located near the surface of a body (e.g., in an artery or vein in the leg, neck, or arm) and maneuvered to a region of interest within the body to enable diagnosis and treatment of tissue without the need for more invasive procedures. For example, ablation catheters are used to convey an electrical stimulus to a region of interest within the body to create tissue necrosis. Ablation catheters may be used to create necrosis in heart tissue to correct conditions such as atrial arrhythmia (including, but not limited to, ectopic atrial tachycardia, atrial fibrillation, and atrial flutter). Arrhythmia can create a variety of dangerous conditions including irregular heart rates, loss of synchronous atrioventricular contractions and statis of blood flow which can lead to a variety of ailments and even death. It is believed that the primary cause of arrhythmia is stray electrical signals within the heart. The ablation catheter imparts ablative energy (e.g., radiofrequency energy) to the heart tissue to create a lesion in the heart tissue. This lesion disrupts electrical pathways and thereby limits or prevents stray electrical signals that lead to arrhythmia.

For proper diagnosis and treatment, it is essential to be able to determine various characteristics of the tissue and/or the catheter during use of the catheter including, for example, the position of the catheter both within the body and relative to the tissue in the region of interest, the contact pressure between the catheter and tissue, and, in the case of ablation catheters, the stage of necrosis in the tissue. In the case of ablation catheters, for example, proper positioning is essential to locating tissue lesions and controlling the depth of the lesions.

Fluoroscopy is one conventional method for both guiding and determining the position of the catheter within a body. In fluoroscopy, a fluoroscope passes a continuous x-ray beam through a body to an imaging device. Fluoroscopes are rather large in size, however, and difficult to maneuver. As a result, it is difficult to accomplish fluoroscopy in multiple planes. Given the complex geometry of an organ like the heart, this limitation can therefore lead to inaccuracies in identifying position. Moreover, fluoroscopy produces prolonged exposure to x-ray radiation—particularly for medical staff that are present for numerous procedures. An electrogram sensed at the distal portion of the catheter may be used together with a fluoroscope to provide a more accurate assessment of catheter position. The arrythmogenic electrical activity within the heart, however, can make signal interpretation difficult.

Another conventional method for guiding catheters is the use of endocardial mapping systems. In these systems, a three-dimensional geometry of the heart chambers is created based on an analysis of electrical signals as the catheter is maneuvered within the chambers. Although this type of system is useful in visualizing the complex macroscopic geometry of the heart chambers and guiding the catheter to a region of interest, the generated image does not provide adequate information regarding the distance from the catheter to the tissue. In particular, the generated image remains static after the geometry is mapped and thereby does not account for changes in position as the heart beats.

It has recently been recognized in U.S. Published Patent Application No. 2006/0229515 A1 that the degree of modification of tissue (e.g., the depth of a lesion formed during ablation) can be monitored using fiber optics. Although a welcome advancement, the system and method described in this application fails to recognize potential additional diagnostic and treatment uses for this type of system. Moreover, the described system and method relies entirely on radiation reflected from a bare tissue wall which limits the information that can be derived.

The inventors herein have recognized a need for a system and method for diagnosis or treatment of tissue that will minimize and/or eliminate one or more of the above-identified deficiencies.

### BRIEF SUMMARY OF THE INVENTION

It is desirable to be able to determine various characteristics of tissue in a body for use in treatment and diagnosis of tissue. For example, it is useful to determine the distance between tissue and the distal end of a catheter during medical procedures. It is also useful to determine the contact pressure between the distal end of a catheter and the tissue to allow for controlled diagnosis and treatment of the tissue. It is also desirable to determine the stage of necrosis in tissue during tissue ablation, the boundaries between tissues of different types, and to identify the type of tissue in a region of interest. It is also desirable to determine the presence or absence of a substance within a region of interest in tissue (e.g., prior to treatment in which the substance is activated) and the condition of the tissue. For use in determining these and other characteristics of tissue, the inventors have developed a system and method for diagnosis and treatment of tissue.

A system in accordance with one aspect of the present invention includes a deformable, tubular body defining a proximal end and a distal end and a first optic fiber disposed within the tubular body. The system further includes an electromagnetic radiation source and an electromagnetic radiation sensor. The system further includes an electronic control unit configured to perform several functions. In particular, the electronic control unit is configured to selectively activate the electromagnetic radiation source to direct a first set of electromagnetic radiation through the first optic fiber to the tissue, the tissue containing a substance that alters radiation characteristics of the tissue. The unit is further configured to receive a signal generated by the electromagnetic radiation sensor in response to a second set of electromagnetic radiation received through one of the first optic fiber and a second optic fiber disposed within the tubular body. The second set of electromagnetic radiation originates from the tissue in response to the first set of electromagnetic radiation. Finally, the control unit is configured to determine a characteristic of the tissue responsive to the signal. The characteristic may comprise, for example, a distance from the tissue to the tubular body, a contact pressure between the tissue and the tubular body, a stage of necrosis of a region of interest in the tissue, a tissue type, a tissue boundary, the presence of the substance in the tissue or a condition of the tissue.

A method in accordance with one aspect of the present invention may include the step of directing a first set of electromagnetic radiation from an electromagnetic radiation source through a first optic fiber disposed within a deformable, tubular body to the tissue, the tissue containing a substance that alters radiation characteristics of the tissue. The method may further include the step of generating a signal responsive to a second set of electromagnetic radiation received through one of the first optic fiber and a second optic fiber disposed within the tubular body, the second set of electromagnetic radiation originating from the tissue in response to the first set of electromagnetic radiation. The method may further include the step of determining a characteristic of the tissue responsive to the signal.

The above-described system and method are advantageous because the use of a substance (e.g., a photodynamic substance) that alters the radiation characteristics of the tissue enables significant additional information to be obtained for use in diagnosis and treatment of the tissue. For example, the substance can allow confirmation of a target site for diagnosis or treatment by enabling differentiation of the target site from other tissue. The substance can also enable confirmation of therapeutic effects. For example, the amount or other characteristics of the substance may change in tissue that has undergone necrosis during an ablation procedure.

A system in accordance with another aspect of the present invention may include a deformable, tubular body defining a proximal end and a distal end and a first optic fiber disposed within the tubular body. The system may further include an electromagnetic radiation source and an electromagnetic radiation sensor. The system may further include an electronic control unit configured to selectively activate the electromagnetic radiation source to direct a first set of electromagnetic radiation through said first optic fiber to the tissue. The unit may further be configured to receive a signal generated by the electromagnetic radiation sensor in response to a second set of electromagnetic radiation received through one of said first optic fiber and a second optic fiber disposed within said tubular body. The second set of electromagnetic radiation may originate from the tissue in response to the first set of electromagnetic radiation. The unit may further be configured to determine a characteristic of the tissue responsive to the signal, the characteristic selected from the group consisting of a distance from the tissue to the tubular body, a contact pressure between the tissue and the tubular body, a stage of necrosis of a region of interest in the tissue, a tissue type, a tissue boundary, a presence of the first substance within the tissue, and a condition of the tissue.

Similarly, a method in accordance with this aspect of the present invention may include the step of directing a first set of electromagnetic radiation from an electromagnetic radiation source through a first optic fiber disposed within a deformable, tubular body to the tissue. The method may further include the step of generating a signal responsive to a second set of electromagnetic radiation received through one of the first optic fiber and a second optic fiber disposed within the tubular body. The second set of electromagnetic radiation may originate from the tissue in response to the first set of electromagnetic radiation. The method may further include the step of determining a characteristic of the tissue responsive to the signal, the characteristic selected from the group consisting of a distance from the tissue to the tubular body, a contact pressure between the tissue and the tubular body, a stage of necrosis of a region of interest in the tissue, a tissue type, a tissue boundary, a presence of said first substance within the tissue, and a condition of the tissue.

The above-described system and method are advantageous relative to conventional methods for determining the distance between the tissue and catheter tip, the contact pressure between the tissue and catheter tip, and other characteristics of the tissue. In particular, the inventive system and method allow determinations of these characteristics and others through improved imagery that is dynamic (rather than static) and that is not subject to interference by electrical sources within the body. Further, the system and method enable determinations to be made with less exposure to potentially harmful forms of radiation.

The foregoing and other aspects, features, details, utilities and advantages of the present invention will be apparent from reading the following description and claims, and from reviewing the accompanying drawings.

### BRIEF DESCRIPTION OF THE DRAWINGS

FIG. 1 is diagrammatic view of a system in accordance with one embodiment of the present invention.

FIG. 2 is a diagrammatic view of system in accordance with another embodiment of the present invention.

FIG. 3 is a diagrammatic view of system in accordance with another embodiment of the present invention.

FIG. 4 is a diagrammatic view of system in accordance with another embodiment of the present invention.

FIG. 5 is a diagrammatic view of system in accordance with another embodiment of the present invention.

### DETAILED DESCRIPTION OF EMBODIMENTS OF THE INVENTION

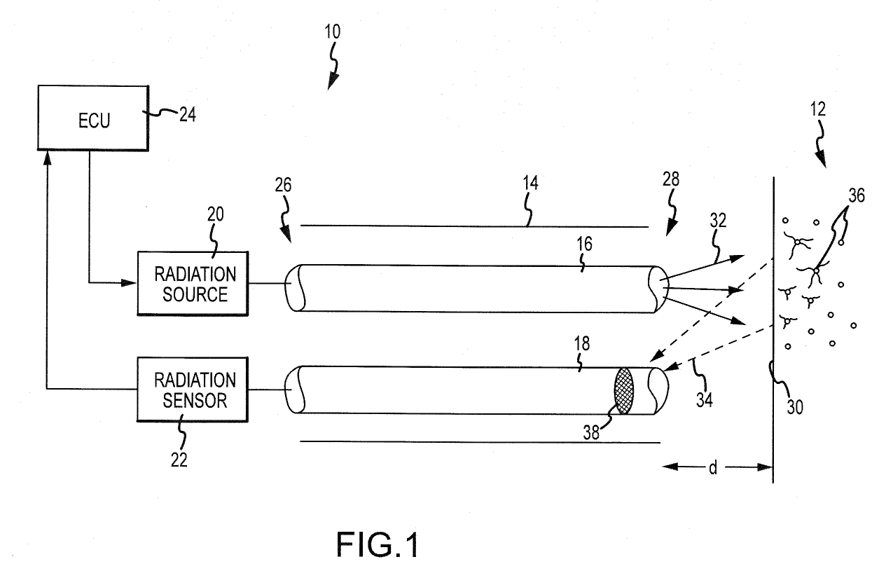

Referring now to the drawings wherein like reference numerals are used to identify identical components in the various views, FIG. 1 illustrates a system 10 for diagnosis and/or treatment of tissue 12 in a body. In one embodiment of the invention, tissue 12 comprises endocardial tissue within the heart of a human body. It should be understood, however, that the inventive system 10 may find application in connection with the diagnosis and treatment of a variety of tissues within human and non-human bodies. System 10 may include a deformable, tubular body 14, a plurality of optic fibers including fibers 16, 18, an electromagnetic radiation source 20, an electromagnetic radiation sensor 22, and an electronic control unit (ECU) 24.

Body 14 functions as a catheter and is provided to house fibers 16, 18. Body 14 may also allow removal of bodily fluids or injection of fluids and medicine into the body. Body 14 may further provide a means for transporting surgical tools or instruments within a body. For example, body 14 may house an electrode (not shown) used in ablation of tissue 12. Body 14 may be formed from conventional materials such as polyurethane. Body 14 is tubular and is deformable and may be guided within a body by a guide wire or other means known in the art. Body 14 has a proximal end 26 and a distal end 28 (as used herein, “proximal” refers to a direction toward the body of a patient and away from the clinician while “distal” refers to a direction toward the clinician and away from the body of a patient). Body 14 may be inserted within a vessel located near the surface of a body (e.g., in an artery or vein in the leg, neck, or arm) in a conventional manner and maneuvered to a region of interest 30 in tissue 12.

Optic fibers 16, 18 are provided to transmit and receive electromagnetic radiation. Fibers 16, 18 are conventional any may be made from various glass compositions (e.g., silica) or plastics (e.g., polymethyl methaacrylate (PMMA) surrounded by fluorinated polymers). Fibers 16, 18 include a core and a cladding with the core having a higher refractive index than the cladding. Fibers 16, 18 may further include a buffer layer and a jacket as is known in the art. Fibers 16, 18 may, for example, comprise any of a variety of common fibers sold by Polymicro Technologies, Inc., Edmund Optics, Inc. or Keyence Corporation. Fibers 16, 18 are disposed within body 14 and may extend from proximal end 26 to distal end 28 of body 14.

Electromagnetic radiation source 20 is provided to generate a set of electromagnetic radiation for transmission through one or more optic fibers. In the illustrated embodiment, source 20 transmits radiation through fiber 16. Source 20 may comprise, for example, a light emitting diode (LED) or laser (e.g., a laser diode). Source 20 may produce a monochromatic or spectral radiation and the radiation may be polarized or unpolarized. Source 20 may generate radiation at various points along the electromagnetic spectrum including, for example, visible light, infrared, near infrared, ultraviolet and near ultraviolet radiation. Radiation source 20 may emit radiation in a controlled manner responsive to signals received from control unit 22. Source 20 may be located at or near the proximal end of fiber 16 and/or proximal end 26 of body 14.

Electromagnetic radiation sensor 22 is provided to generate a signal in response to a set of electromagnetic radiation received through an optic fiber. In the embodiment illustrated in FIG. 1, sensor 22 receives radiation transmitted through fiber 18. In accordance with the present invention, and as discussed in greater detail below, radiation received through fiber 18 originates from tissue 12 in response to radiation transmitted through fiber 16 from source 20. Sensor 22 may comprise a photodiode. Sensor 22 may be located at or near the proximal end of fiber 18 and/or proximal end 26 of body 14.

Electronic control unit (“ECU”) 24 provides a means for selectively activating source 20 to direct a set of electromagnetic radiation through fiber 16 to tissue 12. ECU 24 also provides a means for receiving a signal generated by sensor 22 in response to another set of electromagnetic radiation received through fiber 18 and originating from tissue 12 in response to the radiation transmitted through fiber 16. ECU 24 also provides a means for determining a characteristic of tissue 12 responsive to the signal. ECU 24 may comprise a programmable microprocessor or microcontroller or may comprise an application specific integrated circuit (ASIC). ECU 24 may include a central processing unit (CPU) and an input/output (I/O) interface through which ECU 24 may receive a plurality of input signals including signals generated by sensor 22 and generate a plurality of output signals to convey information regarding characteristics of tissue 12. These output signals may convey information through variation in amplitude of frequency of voltage or current and may, for example, be used to generate images relating to tissue 12. The input and output signals may comprise electrical signals. Alternatively, signals may be transmitted wirelessly in a conventional manner.

In operation, ECU 24 generates one or more signals to selectively activate source 20. In response, source 20 generates a set of electromagnetic radiation (illustrated generally in FIG. 1 by solid arrows 32) that is transmitted through fiber 16 to tissue 12. Another set of electromagnetic radiation (illustrated generally in FIG. 1 by broken line arrows 34) originates at tissue 12 in response to the radiation 32 transmitted through fiber 16. The radiation 34 originating from tissue 12 may comprise a portion of radiation 32 reflected by tissue 12.

In accordance with one aspect of the present invention, a substance 36 may be introduced into tissue 12 that alters the radiation characteristics of tissue 12 before electromagnetic radiation 32 is directed to tissue 12. Substance 36 is provided to generate radiation 34 by reflecting a portion of radiation 32 or by separately emitting radiation in response to radiation 32. In particular, substance 36 may comprise a photodynamic substance that is relatively inert until activated by radiation of a specific wavelength. Upon activation, substance 36 reflects radiation or emits radiation at a different wavelength than the wavelength that activated substance 36. Substance 36 may comprise a photosensitive chemical or drug or other substance. For example, substance 36 may comprise 5-aminolevulinic acid (ALA), meso-tetra-hydroxyphenyl-chlorin (mTHPC), an electrochromic and potentiometric dye such as di-2-ANEPEQ, di-4-ANEPPS or di-8-ANEPPS, neuromodulators such as Acetylcholine, a cardioplegic solution, or a cryocardioplegic solution (e.g., hypothermic saline). Substance 36 may comprise the substance (porfimer sodium) sold by Axcan Pharma Inc. under the registered trademark “PHOTOFRIN” or the substance sold by Scotia Holdings plc under the registered trademark “FOSCAN.” Alternatively, substance 36 may be a radioopaque substance such as the substance sold by Amersham Health AS under the registered trademark “HYPAQUE” or any of a variety of conventional radioopaque dyes. Substance 36 may also comprise a substance that modifies electrical conductivity in tissue 12 such as saline, one of the above-identified photosenitizers or an anti-stenotic agent. When used as part of a treatment or therapy, substance 36 may comprise a cytotoxic chemical.

Substance 36 may alter a variety of radiation characteristics of tissue 12 including the intensity, wavelength, phase, spectrum, speed, optical path, interference, transmission, absorption, reflection, refraction, diffraction, polarization, modulation, scattering, or fluorescence of received radiation 32 and/or generated radiation 34. In addition, substance 36 may alter electrical or biomechanical properties of tissue 12. For example, substance 36 may alter various electrical properties of tissue 12 including activation potential, electrical conductivity, permittivity and permeability. Substance 36 may alter various biomechanical properties of tissue 12 including temperature, thermal conductivity, blood perfusion and partial pressure of oxygen (pO₂). Substance 36 may produce transient (reversible) effects in tissue 12 (e.g. in situations calling for diagnosis or preconditioning of tissue 12 for treatment). Alternatively, substance 36 may produce irreversible effects in tissue 12 (e.g., when substance 36 is used a part of a therapy or treatment such as chemical ablation).

Substance 36 may be introduced into tissue 12 in a variety of ways such that substance 36 is absorbed into the cells in tissue 12 or binds with the cell membranes. For example, substance 36 may be introduced through in-situ delivery, arterial delivery and/or systemic delivery. One method of in-situ delivery may be through electroporation in which a site limited electric shock is used to create an electric field to cause expansion of the cells in tissue 12 for a period of time to allow substance 36 to enter the cells. Alternative methods of in-situ delivery may be by application of an electrical field on substance 36 itself or using acoustic waves (e.g. ultrasound) to break through the tissue boundary. Alternatively, substance 36 may be infused through the artery, such as the coronary artery, to allow perfusion into tissue 12. It should be understood that these methods of introducing substance 36 to tissue 12 are exemplary only and not intended to limit the scope of the invention.

It should be understood that the inventive system and method may also involve use of multiple photodynamic substances 36. For example, diagnosis or treatment may occur in a region of interest having multiple tissue types. Because different tissues react differently to substances 36 (e.g., some tissues are more responsive than others), it may be advantageous to use different substances 36 within the same region of interest. Alternatively, it may be desirable to have multiple substances 36 reflecting of emitting radiation 34 at different wavelengths to, for example, permit definition of a boundary.

Radiation 34 originating from tissue 12 is transmitted through fiber 18 to sensor 22. Sensor 22 generates one or more signals responsive to radiation 34 which are then transmitted to ECU 24. ECU 24 may determine one of more characteristics of tissue 12 responsive to the signals from sensor 22. ECU 24 may determine characteristics of tissue 12 by evaluating changes in various radiation properties, electrical properties, and/or biomechanical properties of tissue 12 as identified hereinabove. ECU 24 may determine characteristics of tissue 12 by comparing one or more of the above-identified properties in pristine tissue and tissue 12 in which a substance 36 has been introduced or by comparing changes in these properties over time in tissue 12.

In one embodiment of the invention, ECU 24 determines a distance d from tissue 12 to body 14 (e.g., to the distal end 28 of body 14). Material between body 14 and tissue 12, such as particulate matter in the blood (e.g., hemoglobin) refracts and scatters radiation 32 emitted from fiber 16. Increased distance between tissue 12 and body 14 increases the amount of matter between the tissue 12 and body 14 in a proportional manner thereby decreasing the amount of radiation 34 returned to fiber 18 and producing an indication of relative distance between tissue 12 and body 14. Similarly, increased distance between tissue 12 and body 14 results in dispersion of light over a wider area of tissue. Because the intensity of the radiation 34 reflected or emitted by substance 36 is proportional to the intensity of radiation 32 impinging on tissue 12 and substance 36, the amount of radiation 34 returned to fiber 18 is indicative of the distance between tissue 12 and body 14.

ECU 24 may determine a wide variety of characteristics of tissue 12 besides the distance between body 14 and a tissue 12. For example, ECU 24 may determine the contract pressure or contact force between body 14 and tissue 12. As body 14 approaches tissue 12, the characteristics of radiation 34 change because a greater amount of radiation 32 is reflected off of tissue 12 rather than by particulate matter (e.g., blood particles) between body 14 and tissue 12 thereby providing an indication of contact with the tissue 12. As body 14 is pressed into tissue 12, additional tissue wraps around the distal end 28 of body 14 providing increased reflection off of the tissue 12 and an indication of contact pressure.

ECU 24 may also determine a stage of necrosis of tissue 12 (such as the atrial or ventricular myocardium or neural ganglion) during ablation of tissue 12. The greater the degree of necrosis, the greater the degree of change in the amount of radiation 34 that will be reflected or emitted by tissue 12.

ECU 24 may also identify different tissue types (including tissue structures) and tissue boundaries between tissues of different types because of the difference in radiation characteristics between different tissues. Within the heart, ECU 24 may determine the tissue type from among various heart tissues such as the pericardium, epicardium, endocardium, myocardium, fossa ovalis, fat pads and blood vessels. ECU 24 may further be used to identify valves, scar tissue, trabeculated tissue and smooth wall tissue.

ECU 24 may also determine the presence or absence of substance 36 in tissue 12. As discussed above, the presence of substance 36 will alter the radiation characteristics of tissue 12. Determining the presence or absence of the substance 36 may be useful in identifying tissues having a certain condition (e.g., because the presence of the substance 36 (and the receptivity of the tissue 12 to substance 36) is indicative of a condition). Determining the presence or absence of substance 36 within tissue 12 is also useful in determining readiness for treatment (e.g., where the substance 36 may be further used for ablation).

ECU 24 may further determine a condition of the tissue 12 indicative of a difference (and potential malformation) relative to surrounding tissue or readiness of a further diagnostic or therapeutic procedure. For example, ECU 24 may determine the presence of patent foramen ovalis (PFO) or a state of tissue perfusion in heart tissue or the presence of scar tissue.

It should be understood that the characteristics described herein are exemplary only and a variety of characteristics may be evaluated using the present invention. Further, it should be understood that ECU 24 may determine the above-described characteristics of tissue 12, and other characteristics, by sensing a variety of parameters associated with electromagnetic radiation including intensity, wavelength, phase, spectrum, speed, optical path, interference, transmission, absorption, reflection, refraction, diffraction, polarization, modulation, scattering and fluorescence.

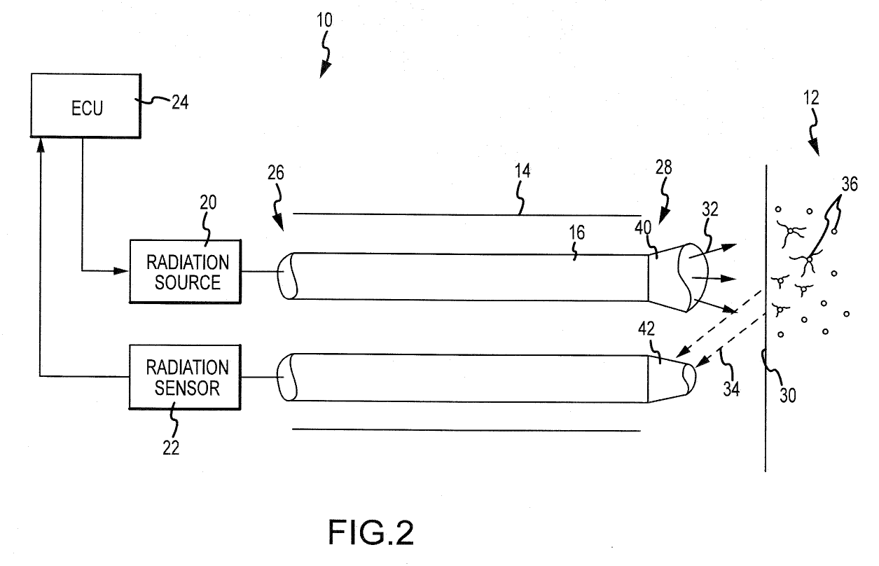

Referring now to FIGS. 1-2, the radiation 32 transmitted by fiber 16 and/or the radiation 34 received by fiber 18 may be controlled or amplified using different components. Referring to FIG. 1, a filter 38 may be disposed within fiber 18 or may cover the proximal or distal end of fiber 18 to control the passage of radiation 34 to sensor 22 by permitting passage of radiation of a selected wavelength (or range of wavelengths) while filtering out radiation 32 and optical noise. Referring to FIG. 2, lenses 40, 42 may be used to focus radiation 32 and/or radiation 34. Lens 40, 42 may be located at the distal end of fibers 16, 18, respectively. It should also be understood that filter 38 and lens 40 or lens 42 may be used together.

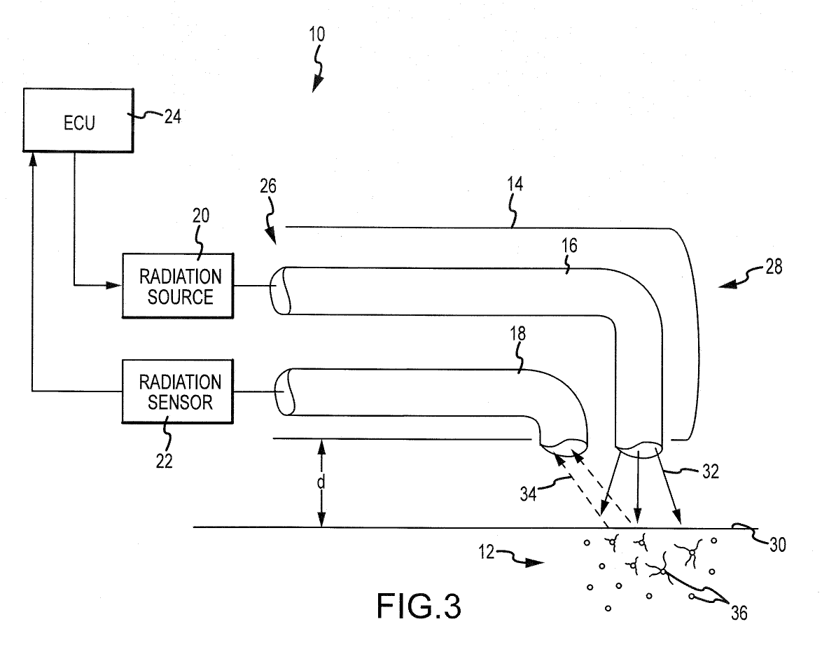

Referring again to FIG. 1, fiber 16 directs radiation 32 and fiber 18 receives radiation 34 through the distal end 28 of body 14. Referring now to FIG. 3, in an alternative embodiment of the invention, fiber 16 directs radiation 32 and fiber 18 receives radiation 34 through a lateral wall 44 between the proximal and distal ends 26, 28 of body 14. The ability to direct and receive radiation through a lateral wall of body 14 is advantageous in certain circumstances. For example, certain applications require that diagnosis and treatment occur in an orientation perpendicular to the longitudinal direction of the catheter. In the case of heart tissue, fiber 16 may be navigated through the body to an endocardial or epicardial surface of the heart or through the esophagus. When diagnosing or treating epicardial tissue the distal end 28 of the body 14 engages the wall of the epicardial sac and diagnosis and treatment must occur through the lateral wall of body 14. Similarly, when body 14 enters the body through the esophagus on the posterior side of the heart, the distal end 28 of body 14 is not oriented towards the heart. Rather, a lateral wall of body 14 faces the heart. In ablation procedures, it can also be desirable to act through the lateral wall of body 14 when producing linear lesions (often used to treat atrial flutter) so that the entire lesion can be formed simultaneously rather than requiring movement of body 14.

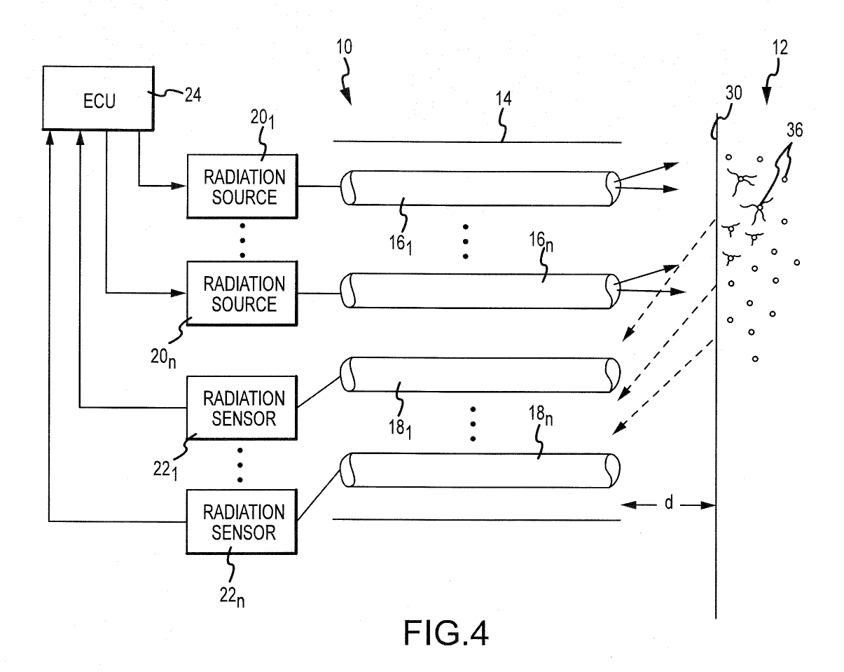

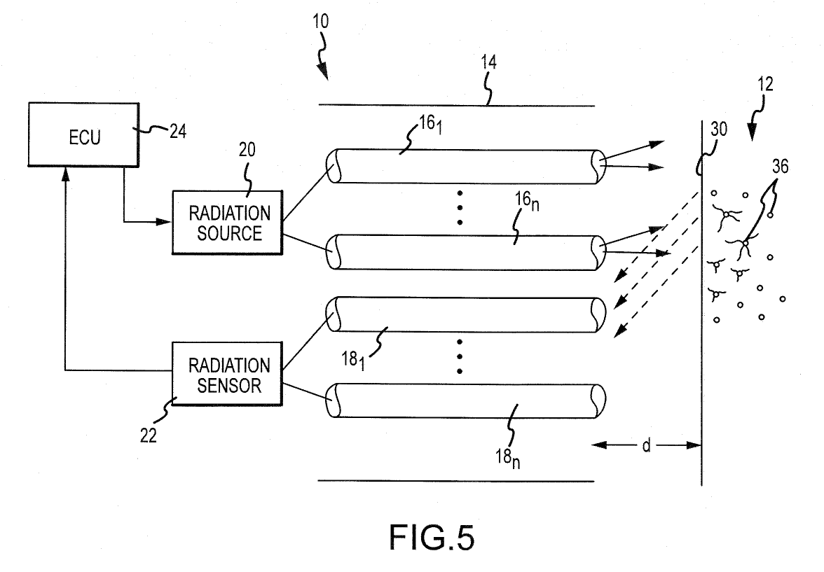

Referring to FIGS. 1-3, embodiments of the invention described thus far illustrate a single radiation source 20 in a one to one relationship with a single fiber 16 as well as a single radiation sensor 22 in a one to one relationship with a single fiber 18. In accordance with other embodiments of the invention, however, both the number of fibers 16, 18, radiation sources 20, and radiation sensors 22 as well as the one to one relationship between the fibers 16, 18 and the source 20 and sensor 22 may vary. Referring to FIG. 4, in another embodiment of the invention, multiple radiation sources 20₁ to 20_{N} and sensors 22₁ to 22_{N} may be used for transmitting radiation and detecting radiation through multiple fibers 16₁ to 16_{N}, 18₁ to 18_{N}, respectively. The use of multiple sources 20 and sensors 22 enables radiation to be transmitted and received using different radiation characteristics (e.g., frequency, intensity, phase angle, polarization) thereby allowing a single system to be used in various applications requiring different characteristics of radiation or allowing for simultaneous use in a single application in which it is desirable to transmit and receive radiation having different characteristics. Referring to FIG. 5, in another embodiment of the invention, a radiation source 20 may transmit radiation 32 through multiple fibers 16₁ to 16_{N} while a sensor 22 receives radiation 34 through one or more fibers 18. It should also be understood that a single fiber 16 may be used to both transmit radiation 32 and receive radiation 34 using a conventional splitter within fiber 16. It should further be understood that the transmitted radiation 32 and received radiation 34 may be directed through one or more fibers 16, 18 in different tubular bodies 14, allowing radiation 32 to be directed through a fiber 16 in one tubular body 14 at one location near tissue 12 (e.g., within the esophagus in the case of heart tissue) and radiation 34 to be received through a fiber 18 in another tubular body at another location near tissue 12 (e.g., within one of the atria in the case of heart tissue). From these examples, it should be understood that the number of fibers 16, 18, radiation sources 20 and radiation sensors 22—as well as the numerical relationship between fibers 16, 18 on the one hand and radiation source 20 and radiation sensor 22 on the other hand—may vary in a number of ways depending on the ultimate application.

A system and method in accordance with the present invention offers a number of advantages. In particular, the system and method enable a more accurate determination of various characteristics of tissue in a region of interest. For example, the inventive system and method provide a more accurate assessment of the position of a catheter relative to a tissue than fluoroscopy without requiring significant exposure to radiation associate with fluoroscopy. The inventive system reduces signal noise from areas outside the region of interest because radiation 34 that is received is generated in response to the targeted delivery of radiation 32. The inventive system also is capable of use during ablation procedures because the types of electromagnetic radiation that are likely to be used (from ultraviolet to infrared) will prevent interference with, or distortion by, common ablation energy modalities such as radio frequency waves or microwaves. The use of a substance that alters the radiation characteristics of the tissue in certain embodiment of the invention also provides significant advantages relative to conventional methods for tissue diagnosis and treatment. Use of the substance permits confirmation of target sites for treatment and diagnosis as well as confirmation of therapeutic effects.

Although several embodiments of this invention have been described above with a certain degree of particularity, those skilled in the art could make numerous alterations to the disclose embodiments without departing from the spirit or scope of this invention. All directional references (e.g., upper, lower, upward, downward, left, right, leftward, rightward, top, bottom, above, below, vertical, horizontal, clockwise and counterclockwise) are only use for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not as limiting. Changes in detail or structure may be made without departing from the spirit of the invention as defined in the appended claims.

1. A system for diagnosis or treatment of a tissue in a body, comprising:
a deformable, tubular body defining a proximal end and a distal end;
a first optic fiber disposed within said tubular body;
a first electromagnetic radiation source;
an electromagnetic radiation sensor; and
an electronic control unit configured to:
selectively activate said first electromagnetic radiation source to direct a first set of electromagnetic radiation through said first optic fiber to said tissue, said tissue containing a first substance that alters radiation characteristics of said tissue;
receive a signal generated by said electromagnetic radiation sensor in response to a second set of electromagnetic radiation received through one of said first optic fiber and a second optic fiber disposed within said tubular body, said second set of electromagnetic radiation originating from said tissue in response to said first set of electromagnetic radiation; and
determine a first characteristic of said tissue responsive to said signal.

2. The system of claim 1 wherein said second set of electromagnetic radiation comprises a portion of said first set of electromagnetic radiation reflected from said tissue.

3. The system of claim 1 wherein said first substance comprises a photodynamic substance and second set of electromagnetic radiation comprises a portion of said first set of electromagnetic radiation reflected by said photodynamic substance.

4. The system of claim 1 wherein said first substance comprises a photodynamic substance and said second set of electromagnetic radiation comprises electromagnetic radiation emitted by said photodynamic substance.

5. The system of claim 1, further comprising a filter disposed within said one optic fiber.

6. The system of claim 1, further comprising a focusing lens disposed in one of said first and second optic fibers.

7. The system of claim 1 wherein said first set of electromagnetic radiation is directed, and said second set of electromagnetic radiation is received, through said distal end of said tubular body.

8. The system of claim 1 wherein said first set of electromagnetic radiation is directed, and said second set of electromagnetic radiation is received, through a lateral wall of said tubular body between said proximal and distal ends of said tubular body.

9. (canceled)

10. The system of claim 1, further comprising a second electromagnetic radiation source, said electronic control unit further configured to selectively activate said second electromagnetic radiation source to thereby direct a third set of electromagnetic radiation through a third optic fiber disposed within said tubular body to said tissue, said third set of electromagnetic radiation having different radiation characteristics than said first set of electromagnetic radiation.

11. The system of claim 1 wherein said first characteristic includes at least one of a distance from said tissue to said tubular body, a contact pressure between said tissue and said tubular body, a stage of necrosis of a region of interest in said tissue, a tissue type, a tissue boundary, and a presence of said first substance within said tissue.

12. (canceled)

13. (canceled)

14. (canceled)

15. (canceled)

16. (canceled)

17. (canceled)

18. The system of claim 1 wherein said radiation characteristics include at least one of frequency of radiation, intensity of radiation, phase angle of radiation and polarization of radiation.

19. A method for diagnosis or treatment of a tissue in a body, comprising the steps of:
directing a first set of electromagnetic radiation from a first electromagnetic radiation source through a first optic fiber disposed within a deformable, tubular body to a tissue containing a first substance that alters radiation characteristics of said tissue;
generating a signal responsive to a second set of electromagnetic radiation received through one of said first optic fiber and a second optic fiber disposed within said tubular body, said second set of electromagnetic radiation originating from said tissue in response to said first set of electromagnetic radiation; and
determining a first characteristic of said tissue responsive to said signal.

20. The method of claim 19 wherein said second set of electromagnetic radiation comprises a portion of said first set of electromagnetic radiation reflected from said tissue.

21. (canceled)

22. The method of claim 19 wherein said first substance comprises a photodynamic substance and said second set of electromagnetic radiation comprises a portion of said first set of electromagnetic radiation reflected by said photodynamic substance.

23. The method of claim 19 wherein said first substance comprises a photodynamic substance and said second set of electromagnetic radiation comprises electromagnetic radiation emitted by said photodynamic substance.

24-35. (canceled)

36. A system for diagnosis or treatment of a tissue in a body, comprising:
a deformable, tubular body defining a proximal end and a distal end;
a first optic fiber disposed within said tubular body;
a first electromagnetic radiation source;
an electromagnetic radiation sensor; and
an electronic control unit configured to:
selectively activate said first electromagnetic radiation source to direct a first set of electromagnetic radiation through said first optic fiber to said tissue;
receive a signal generated by said electromagnetic radiation sensor in response to a second set of electromagnetic radiation received through one of said first optic fiber and a second optic fiber disposed within said tubular body, said second set of electromagnetic radiation originating from said tissue in response to said first set of electromagnetic radiation; and
determine a characteristic of said tissue responsive to said signal, said characteristic selected from the group consisting of a distance from said tissue to said tubular body, a contact pressure between said tissue and said tubular body, a stage of necrosis of a region of interest in said tissue, a tissue type, a tissue boundary, a presence of said first substance within said tissue, and a condition of said tissue.

37-46. (canceled)

47. The system of claim 36, further comprising a second electromagnetic radiation source, said electronic control unit further configured to selectively activate said second electromagnetic radiation source to thereby direct a third set of electromagnetic radiation through a third optic fiber disposed within said tubular body to said tissue, said third set of electromagnetic radiation having different radiation characteristics than said first set of electromagnetic radiation.

48-56. (canceled)
